(19) United States
(12) Patent Application Publication
(10) Pub. No.: US 2008/0269775 A1
(43) Pub. Date: Oct. 30, 2008

(54) Method and apparatus for providing medical treatment using visualization components of audio spectrum signals

(76) Inventor: Kenneth R. Lemons, Indianapolis, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/148,614

(22) Filed: Apr. 21, 2008

Related U.S. Application Data
(60) Provisional application No. 60/913,052, filed on Apr. 20, 2007.
(60) Provisional application No. 61/025,542, filed on Feb. 1, 2008.

(51) Int. Cl. A61B 8/00

(52) U.S. Cl. 606/127

(57) ABSTRACT

The present disclosure relates to medical imaging and treatment devices and methods. A system is provided that provides location and treatment of kidney stones or other entities through the use of visual representation of audio signals.

### CROSS-REFERENCE TO RELATED APPLICATIONS

The present application claims the benefit of U.S. Provisional Patent Application Ser. No. 60/913,052, filed Apr. 20, 2007, entitled “Method and Apparatus for Providing Medical Treatment Using Visualization Components of Audio Spectrum Signals” and U.S. Provisional Patent Application Ser. No. 61/025,542 filed Feb. 1, 2008 entitled “Apparatus and Method of Displaying Infinitely Small Divisions of Measurement.” This application also relates to U.S. Provisional Patent Application Ser. No. 60/830,386 filed Jul. 12, 2006 entitled “Apparatus and Method for Visualizing Musical Notation”, United States Utility patent application Ser. No. 11/827,264 filed Jul. 11, 2007 entitled “Apparatus and Method for Visualizing Music and Other Sounds”, U.S. Provisional Patent Application Ser. No. 60/921,578, filed Apr. 3, 2007, entitled “Device and Method for Visualizing Musical Rhythmic Structures”, and United States Utility patent application Ser. No. 12/023,375 filed Jan. 31, 2008 entitled “Device and Method for Visualizing Musical Rhythmic Structures”. All of these applications are hereby incorporated by reference in their entirety.

### TECHNICAL FIELD OF THE DISCLOSURE

The present disclosure relates generally to medical imaging and treatment systems and, more specifically, to a system and method for providing medical treatment, such as for locating and treating kidney stones, using analysis of tonal and rhythmic structures.

### BACKGROUND OF THE DISCLOSURE

High energy audio frequency or ultrasound signals are used to treat kidney stones in a process called extracorporeal shock wave lithotripsy. In this process, however, X-rays are required to locate the stone so that the high energy signals can be accurately targeted. Although prolonged or accumulated doses of X-radiation is unhealthy, other methods for locating and treating kidney stones have not been commercially developed. Methods and devices are needed which improve the accuracy and efficiency of processes used to locate and treat entities, such as kidneys stones or tumors, within the human body.

### SUMMARY OF THE INVENTION

Accordingly, in one aspect, a system for providing medical treatment of kidney stones, comprising a signal output source; a signal input device; a processing device; and a display, wherein: said signal output source directs a first output signal in the vicinity of an entity in a human body desired to be treated; wherein: said processing device executes computer readable code to create a first visual representation of a first signal reflected by said entity and received by said signal input device for output on said display; and wherein: said first visual representation is generated according to a method comprising the steps of: (a) labeling the perimeter of a circle with a plurality of labels corresponding to a plurality of frequency bands, such that moving radially inward or outward from any one of said labels represents a change in a signal amplitude at the frequency corresponding to said one of first labels; (b) identifying a first occurrence of a first frequency having a first amplitude within said first signal; and (c) graphically indicating a point along a radial axis corresponding to said first amplitude; said radial axis connecting the center of said circle and said first label.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of a twelve-tone circle according to one embodiment.

FIG. 2 is a diagram of a twelve-tone circle showing the six intervals.

FIG. 3 is a diagram of a twelve-tone circle showing the chromatic scale.

FIG. 4 is a diagram of a twelve-tone circle showing the first through third diminished scales.

FIG. 5 is a diagram of a twelve-tone circle showing all six tri-tones.

FIG. 6 is a diagram of a twelve-tone circle showing a major triad.

FIG. 7 is a diagram of a twelve-tone circle showing a major seventh chord.

FIG. 8 is a diagram of a twelve-tone circle showing a major scale.

FIGS. 9-10 are diagrams of a helix showing a B diminished seventh chord.

FIG. 11 is a diagram of a helix showing an F minor triad covering three octaves.

FIG. 12 is a perspective view of the visual representation of percussive music according to one embodiment shown with associated standard notation for the same percussive music.

FIG. 13 is a two dimensional view looking along the time line of a visual representation of percussive music at an instant when six percussive instruments are being simultaneously sounded.

FIG. 14 is a two dimensional view looking perpendicular to the time line of the visual representation of percussive music according to the disclosure associated with standard notation for the same percussive music of FIG. 12.

FIG. 15 is a schematic block diagram showing a system for providing medical treatment according to one embodiment.

FIG. 16 depicts a visualization scheme for displaying visualizations of various frequency amplitudes within a received signal according to one embodiment.

FIG. 17 is an example of a displayed visualization for one frequency component of a received signal according to the scheme of FIG. 16.

FIG. 18 is an example of a displayed combined visualization for a multi-frequency audio signal according to one embodiment.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

Before describing the system and method for medical treatment, a summary of the above-referenced music tonal and rhythmic visualization methods will be presented. The tonal visualization methods are described in U.S. patent application Ser. No. 11/827,264 filed Jul. 11, 2007 entitled “Apparatus and Method for Visualizing Music and Other Sounds” which is hereby incorporated by reference in its entirety.

There are three traditional scales or ‘patterns’ of musical tone that have developed over the centuries. These three scales, each made up of seven notes, have become the foundation for virtually all musical education in the modern world. There are, of course, other scales, and it is possible to create any arbitrary pattern of notes that one may desire; but the vast majority of musical sound can still be traced back to these three primary scales.

Each of the three main scales is a lopsided conglomeration of seven intervals:

Major scale: 2 steps, 2 steps, 1 step, 2 steps, 2 steps, 2 steps, 1 step

### Harmonic Minor Scale: 2, 1, 2, 2, 1, 3, 1

### Melodic Minor Scale: 2, 1, 2, 2, 2, 2, 1

Unfortunately, our traditional musical notation system has also been based upon the use of seven letters (or note names) to correspond with the seven notes of the scale: A, B, C, D, E, F and G. The problem is that, depending on which of the three scales one is using, there are actually twelve possible tones to choose from in the ‘pool’ of notes used by the three scales. Because of this discrepancy, the traditional system of musical notation has been inherently lopsided at its root.

With a circle of twelve tones and only seven note names, there are (of course) five missing note names. To compensate, the traditional system of music notation uses a somewhat arbitrary system of ‘sharps’ (#'s) and ‘flats’ (b's) to cover the remaining five tones so that a single notation system can be used to encompass all three scales. For example, certain key signatures will have seven ‘pure letter’ tones (like ‘A’) in addition to sharp or flat tones (like C^{#} or G^{b}), depending on the key signature. This leads to a complex system of reading and writing notes on a staff, where one has to mentally juggle a key signature with various accidentals (sharps and flats) that are then added one note at a time. The result is that the seven-note scale, which is a lopsided entity, is presented as a straight line on the traditional musical notation staff. On the other hand, truly symmetrical patterns (such as the chromatic scale) are represented in a lopsided manner on the traditional musical staff. All of this inefficiency stems from the inherent flaw of the traditional written system being based upon the seven note scales instead of the twelve-tone circle.

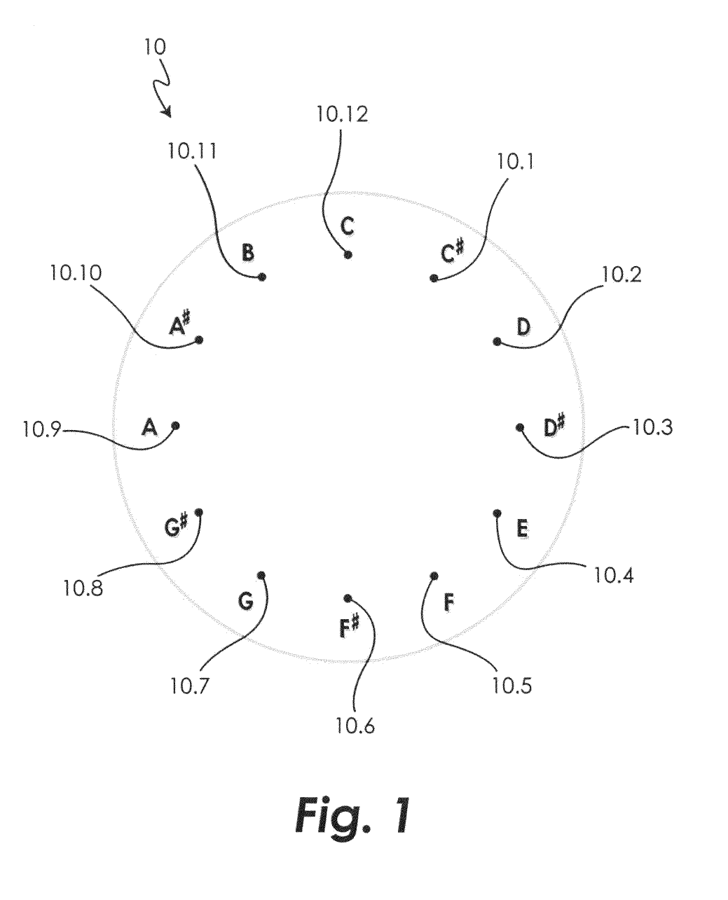

To overcome this inefficiency, a set of mathematically based, color-coded MASTER KEY™ diagrams is presented to better explain the theory and structures of music using geometric form and the color spectrum. As shown in FIG. 1, the twelve tone circle 10 is the template upon which all of the other diagrams are built. Twelve points 10.1-10.12 are geometrically placed in equal intervals around the perimeter of the circle 10 in the manner of a clock; twelve points, each thirty degrees apart. Each of the points 10.1-10.12 on the circle 10 represents one of the twelve pitches. The names of the various pitches can then be plotted around the circle 10. It will be appreciated that in traditional musical notation there are more than one name for each pitch (e.g., A^{#} is the same as B^{b}), which causes inefficiency and confusion since each note can be ‘spelled’ in two different ways. In the illustrated embodiment, the circle 10 has retained these traditional labels, although the present disclosure comprehends that alternative labels can be used, such as the letters A-L, or numbers 1-12. Furthermore, the circle 10 of FIG. 1 uses the sharp notes as labels; however, it will be understood that some or all of these sharp notes can be labeled with their flat equivalents and that some of the non-sharp and non-flat notes can be labeled with the sharp or flat equivalents.

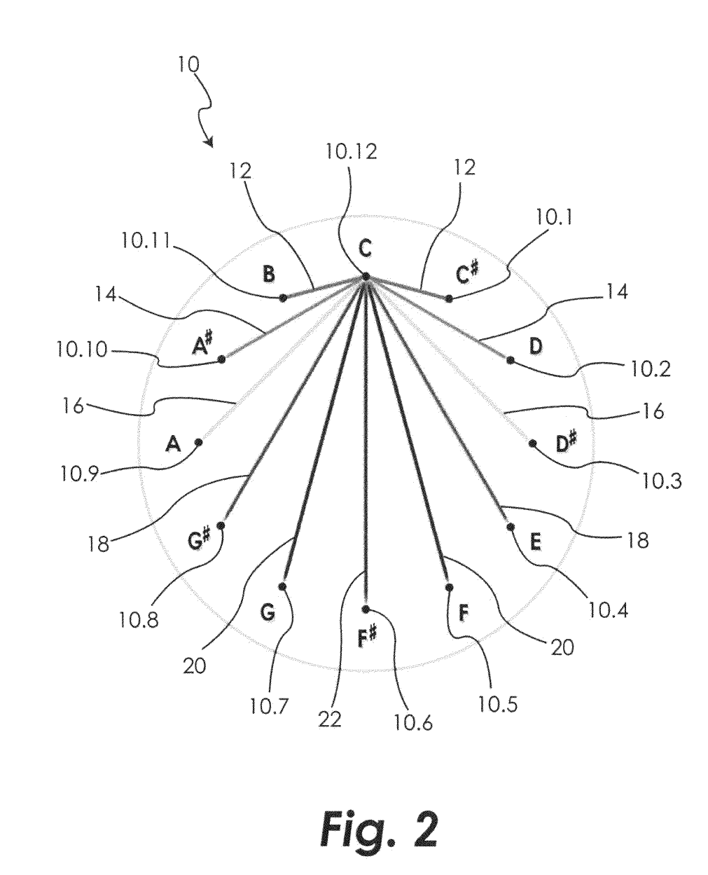

The next ‘generation’ of the MASTER KEY™ diagrams involves thinking in terms of two note ‘intervals.’ The Interval diagram, shown in FIG. 2, is the second of the MASTER KEY™ diagrams, and is formed by connecting the top point 10.12 of the twelve-tone circle 10 to every other point 10.1-10.11. The ensuing lines—their relative length and color—represent the various ‘intervals.’ It shall be understood that while eleven intervals are illustrated in FIG. 2, there are actually only six basic intervals to consider. This is because any interval larger than the tri-tone (displayed in purple in FIG. 2) has a ‘mirror’ interval on the opposite side of the circle. For example, the whole-step interval between C (point 10.12) and D (point 10.2) is equal to that between C (point 10.12) and A^{#} (point 10.10).

Another important aspect of the MASTER KEY™ diagrams is the use of color. Because there are six basic music intervals, the six basic colors of the rainbow can be used to provide another way to comprehend the basic structures of music. In a preferred embodiment, the interval line 12 for a half step is colored red, the interval line 14 for a whole step is colored orange, the interval line 16 for a minor third is colored yellow, the interval line 18 for a major third is colored green, the interval line 20 for a perfect fourth is colored blue, and the interval line 22 for a tri-tone is colored purple. In other embodiments, different color schemes may be employed. What is desirable is that there is a gradated color spectrum assigned to the intervals so that they may be distinguished from one another by the use of color, which the human eye can detect and process very quickly.

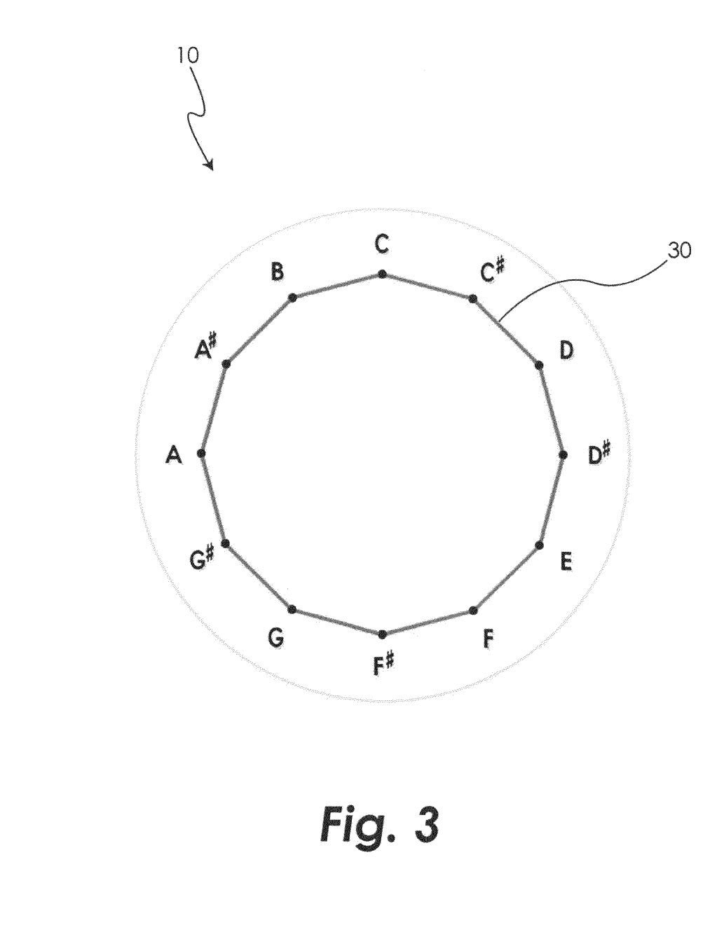

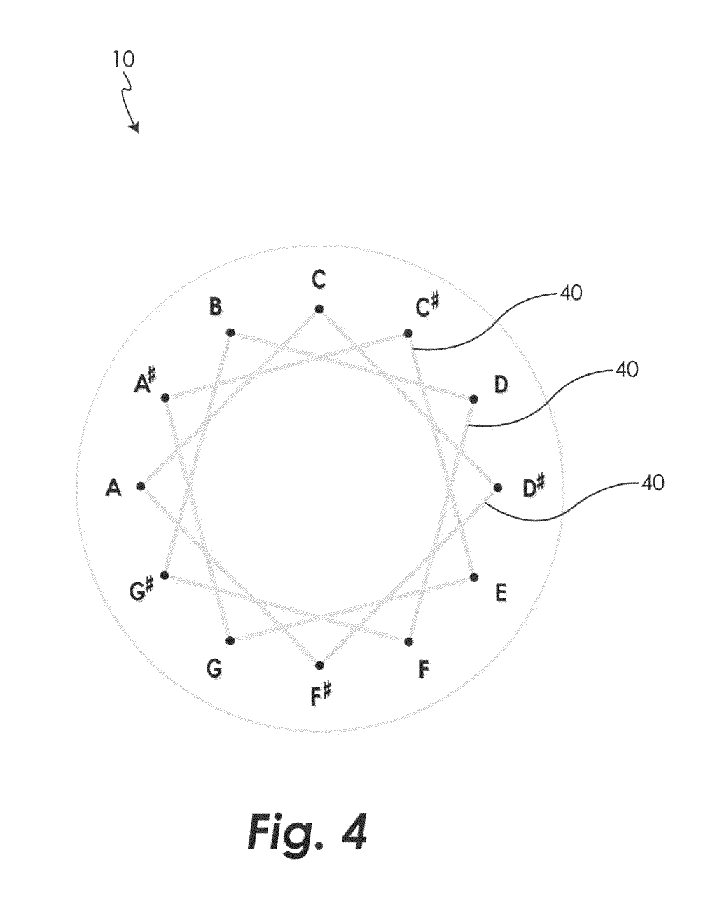

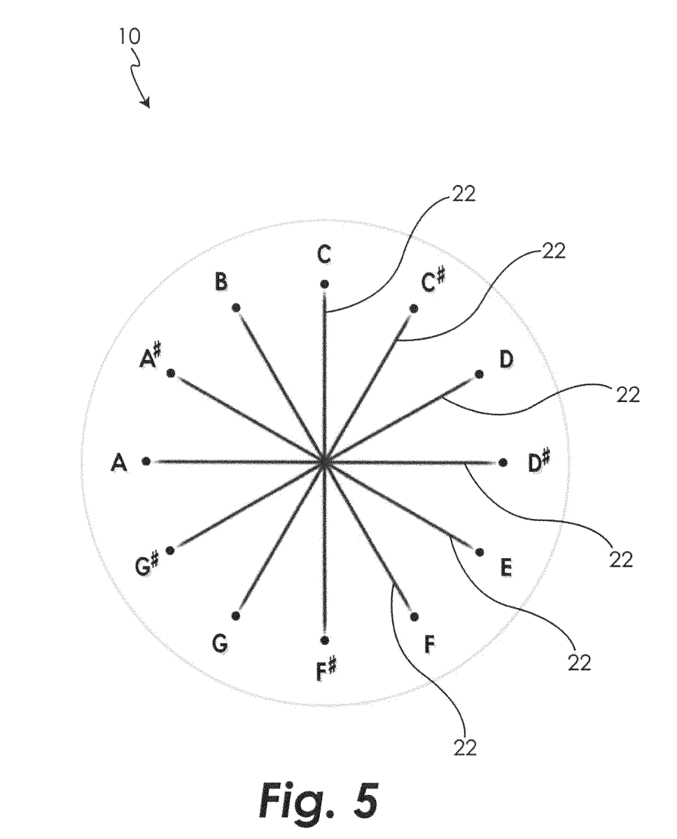

The next group of MASTER KEY™ diagrams pertains to extending the various intervals 12-22 to their completion around the twelve-tone circle 10. This concept is illustrated in FIG. 3, which is the diagram of the chromatic scale. In these diagrams, each interval is the same color since all of the intervals are equal (in this case, a half-step). In the larger intervals, only a subset of the available tones is used to complete one trip around the circle. For example, the minor-third scale, which gives the sound of a diminished scale and forms the shape of a square 40, requires three transposed scales to fill all of the available tones, as illustrated in FIG. 4. The largest interval, the tri-tone, actually remains a two-note shape 22, with six intervals needed to complete the circle, as shown in FIG. 5.

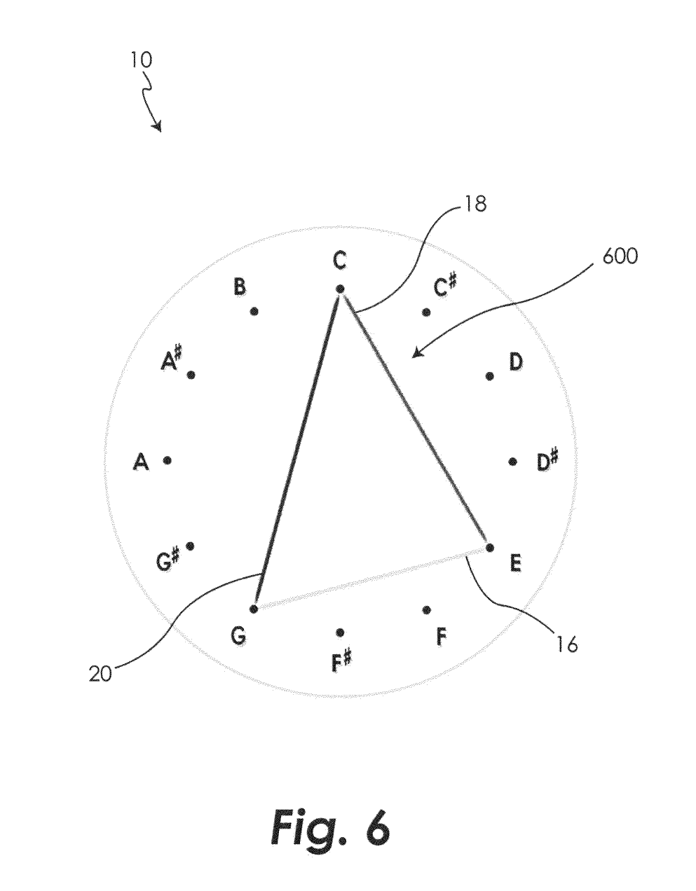

The next generation of MASTER KEY™ diagrams is based upon musical shapes that are built with three notes. In musical terms, three note structures are referred to as triads. There are only four triads in all of diatonic music, and they have the respective names of major, minor, diminished, and augmented. These four, three-note shapes are represented in the MASTER KEY™ diagrams as different sized triangles, each built with various color coded intervals. As shown in FIG. 6, for example, the major triad 600 is built by stacking (in a clockwise direction) a major third 18, a minor third 16, and then a perfect fourth 20. This results in a triangle with three sides in the respective colors of green, yellow, and blue, following the assigned color for each interval in the triad. The diagrams for the remaining triads (minor, diminished, and augmented) follow a similar approach.

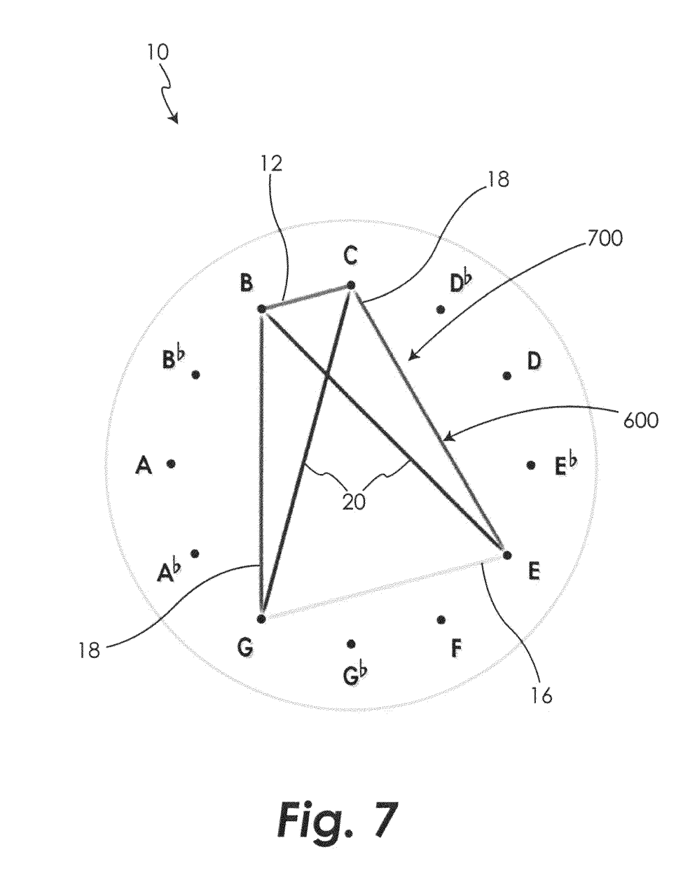

The next group of MASTER KEY™ diagrams are developed from four notes at a time. Four note chords, in music, are referred to as seventh chords, and there are nine types of seventh chords. FIG. 7 shows the diagram of the first seventh chord, the major seventh chord 700, which is created by stacking the following intervals (as always, in a clockwise manner): a major third, a minor third 16, another major third 18, and a half step 12. The above description illustrates the outer shell of the major seventh chord 700 (a four-sided polyhedron); however, general observation will quickly reveal a new pair of ‘internal’ intervals, which haven't been seen in previous diagrams (in this instance, two perfect fourths 20). The eight remaining types of seventh chords can likewise be mapped on the MASTER KEY™ circle using this method.

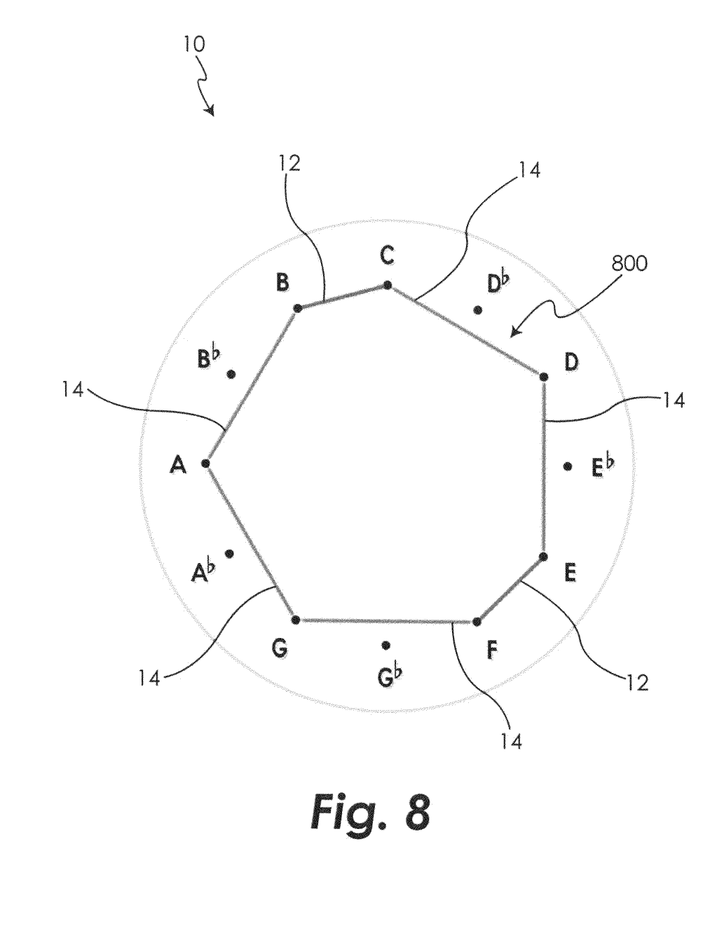

Every musical structure that has been presented thus far in the MASTER KEY™ system, aside from the six basic intervals, has come directly out of three main scales. Again, the three main scales are as follows: the Major Scale, the Harmonic-Minor Scale, and the Melodic-Minor Scale. The major scale is the most common of the three main scales and is heard virtually every time music is played or listened to in the western world. As shown in FIG. 8 and indicated generally at 800, the MASTER KEY™ diagram clearly shows the major scale's 800 makeup and its naturally lopsided nature. Starting at the top of the circle 10, one travels clockwise around the scale's outer shell. The following pattern of intervals is then encountered: whole step 14, whole step 14, half step 12, whole step 14, whole step 14, whole step 14, half step 12. The most important aspect of each scale diagram is, without a doubt, the diagram's outer ‘shell.’ Therefore, the various internal intervals in the scale's interior are not shown. Since we started at point 10.12, or C, the scale 800 is the C major scale. Other major scales may be created by starting at one of the other notes on the twelve-tone circle 10. This same method can be used to create diagrams for the harmonic minor and melodic minor scales as well.

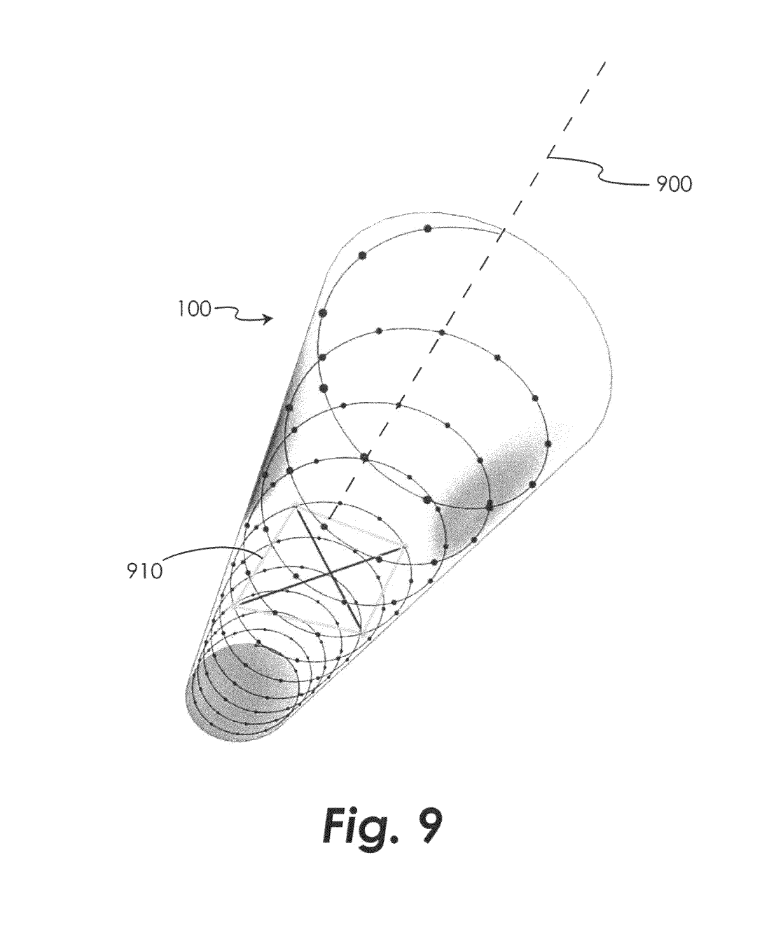

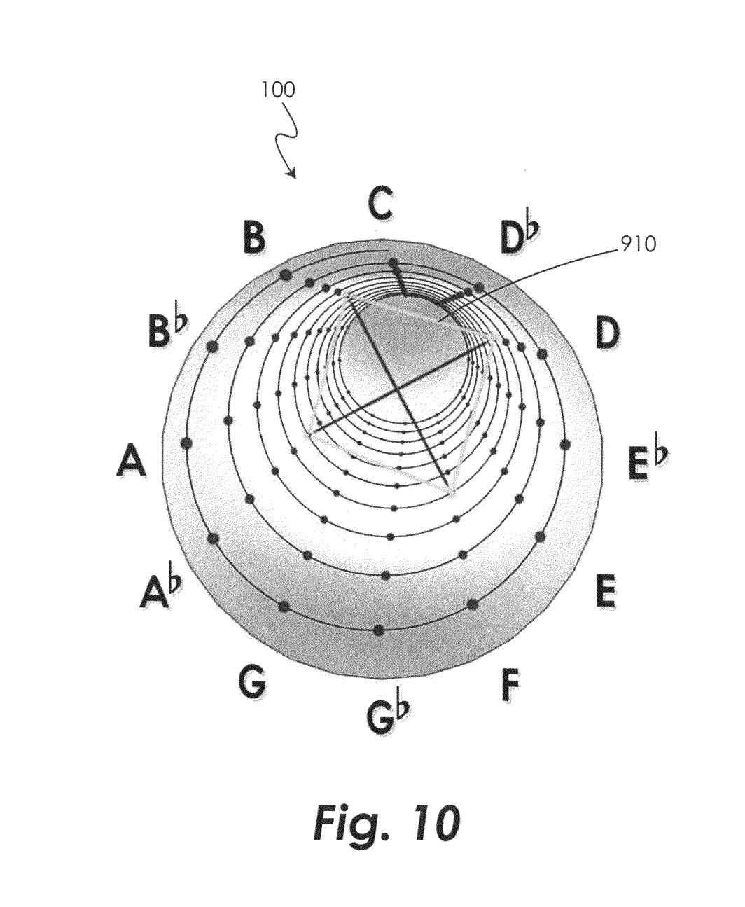

The previously described diagrams have been shown in two dimensions; however, music is not a circle as much as it is a helix. Every twelfth note (an octave) is one helix turn higher or lower than the preceding level. What this means is that music can be viewed not only as a circle but as something that will look very much like a DNA helix, specifically, a helix of approximately ten and one-half turns (i.e. octaves). There are only a small number of helix turns in the complete spectrum of audible sound; from the lowest auditory sound to the highest auditory sound. By using a helix instead of a circle, not only can the relative pitch difference between the notes be discerned, but the absolute pitch of the notes can be seen as well. For example, FIG. 9 shows a helix 100 about an axis 900 in a perspective view with a chord 910 (a fully diminished seventh chord in this case) placed within. In FIG. 10, the perspective has been changed to allow each octave point on consecutive turns of the helix to line up. This makes it possible to use a single set of labels around the helix. The user is then able to see that this is a B fully diminished seventh chord and discern which octave the chord resides in.

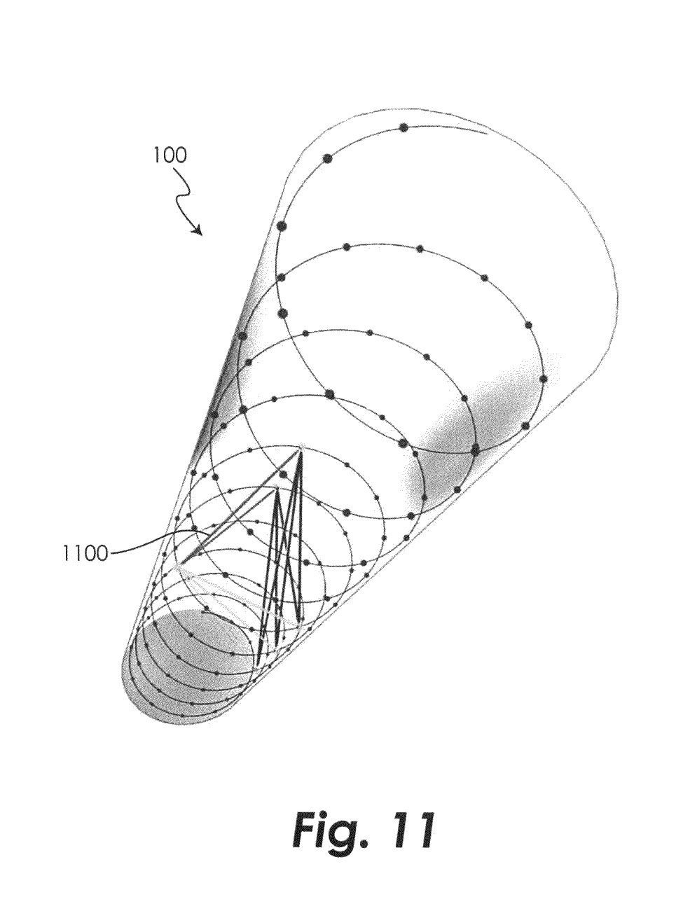

The use of the helix becomes even more powerful when a single chord is repeated over multiple octaves. For example, FIG. 11 shows how three F minor triad chords look when played together over three and one-half octaves. In two dimensions, the user will only see one triad, since all three of the triads perfectly overlap on the circle. In the three-dimensional helix, however, the extended scale is visible across all three octaves.

The above described MASTER KEY™ system provides a method for understanding the tonal information within musical compositions. Another method, however, is needed to deal with the rhythmic information, that is, the duration of each of the notes and relative time therebetween. Such rhythmic visualization methods are described in United States Utility patent application Ser. No. 12/023,375 filed Jan. 31, 2008 entitled “Device and Method for Visualizing Musical Rhythmic Structures” which is also hereby incorporated by reference in its entirety.

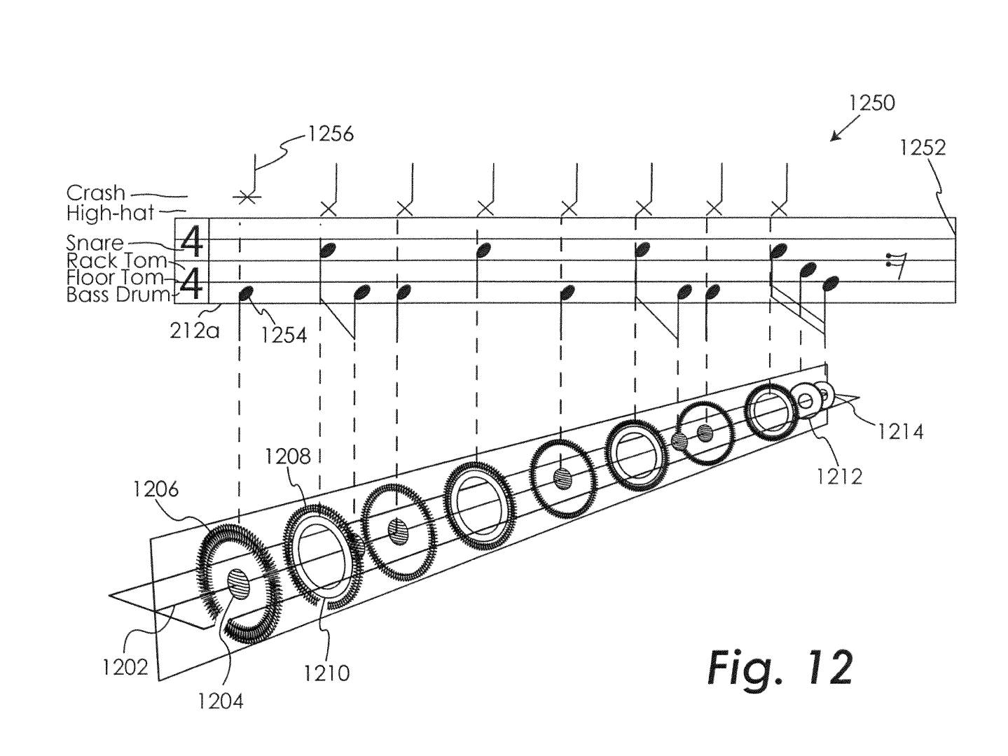

In addition to being flawed in relation to tonal expression, traditional sheet music also has shortcomings with regards to rhythmic information. This becomes especially problematic for percussion instruments that, while tuned to a general frequency range, primarily contribute to the rhythmic structure of music. For example, traditional staff notation 1250, as shown in the upper portion of FIG. 12, uses notes 1254 of basically the same shape (an oval) for all of the drums in a modern drum kit and a single shape 1256 (an ‘x’ shape) for all of the cymbals. What is needed is a method that more intuitively conveys the character of individual rhythmic instruments and the underlying rhythmic structures present in a given composition.

The lower portion of FIG. 12 shows one embodiment of the disclosed method which utilizes spheroids 1204 and toroids 1206, 1208, 1210, 1212 and 1214 of various shapes and sizes in three dimensions placed along a time line 1202 to represent the various rhythmic components of a particular musical composition. The lowest frequencies or lowest instrument in the composition (i.e. the bass drum) will appear as spheroids 1204. As the rhythmical frequencies get higher in range, toroids 1206, 1208, 1210, 1212 and 1214 of various sizes are used to represent the sounded instrument. While the diameter and thicknesses of these spheroids and toroids may be adjustable components that are customizable by the user, the focus will primarily be on making the visualization as “crisply” precise as possible. In general, therefore, as the relative frequency of the sounded instrument increases, the maximum diameter of the spheroid or toroid used to depict the sounding of the instrument also increases. For example, the bass drum is represented by a small spheroid 1204, the floor tom by toroid 1212, the rack tom by toroid 1214, the snare by toroid 1210, the high-hat cymbal by toroid 1208, and the crash cymbal by toroid 1206. Those skilled in the art will recognize that other geometric shapes may be utilized to represent the sounds of the instruments within the scope of the disclosure.

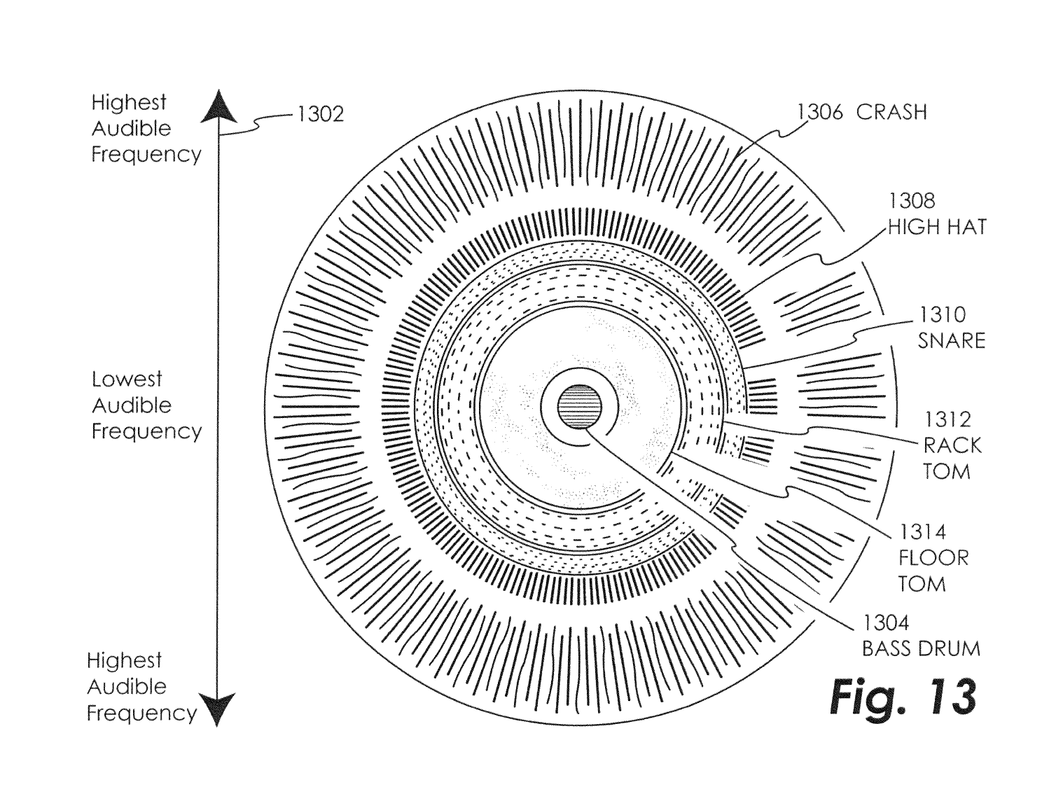

FIG. 13 shows another embodiment which utilizes a two-dimensional view looking into the time line 1202. In this embodiment, the spheroids 1204 and toroids 1206, 1208, 1210 and 1212 from FIG. 12 correspond to circles 1304 and rings 1306, 1308, 1310 and 1312, respectively. The lowest frequencies (i.e. the bass drum) will appear as a solid circle 1304 in a hard copy embodiment. Again, as the relative frequency of the sounded instrument increases, the maximum diameter of the circle or ring used to depict the sounding of the instrument also increases, as shown by the scale 1302.

Because cymbals have a higher auditory frequency than drums, cymbal toroids have a resultantly larger diameter than any of the drums. Furthermore, the amorphous sound of a cymbal will, as opposed to the crisp sound of a snare, be visualized as a ring of varying thickness, much like the rings of a planet or a moon. The “splash” of the cymbal can then be animated as a shimmering effect within this toroid. In one embodiment, the shimmering effect can be achieved by randomly varying the thickness of the toroid at different points over the circumference of the toroid during the time period in which the cymbal is being sounded as shown by toroid 1204 and ring 1306 in FIGS. 12 and 13, respectively. It shall be understood by those with skill in the art that other forms of image manipulation may be used to achieve this shimmer effect.

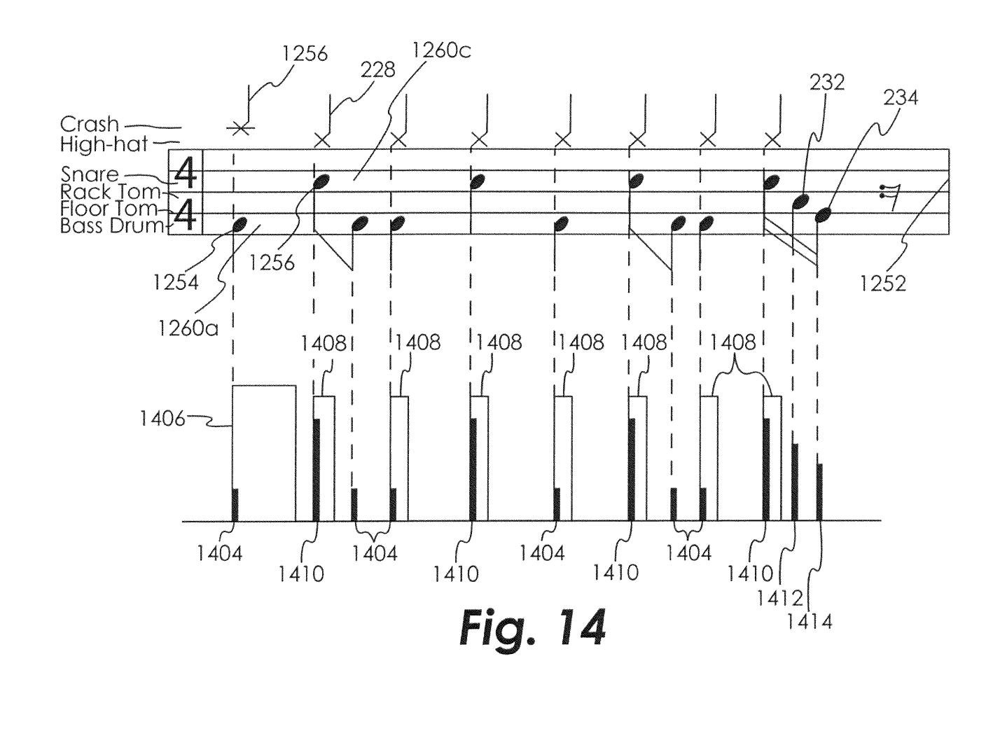

FIG. 14 shows another embodiment which utilizes a two dimensional view taken perpendicular to the time line 1202. In this view, the previously seen circles, spheroids, rings or toroids turn into bars of various height and thickness. Spheroids 1204 and toroids 1206, 1208, 1210, 1212 and 1214 from FIG. 12 correspond to bars 1404, 1406, 1408, 1410, 1412, and 1414 in FIG. 14. For each instrument, its corresponding bar has a height that relates to the particular space or line in, above, or below the staff on which the musical notation for that instrument is transcribed in standard notation. Additionally, the thickness of the bar for each instrument corresponds with the duration or decay time of the sound played by that instrument. For example, bar 1406 is much wider than bar 1404, demonstrating the difference in duration when a bass drum and a crash cymbal are struck. To enhance the visual effect when multiple instruments are played simultaneously, certain bars may be filled in with color or left open.

The spatial layout of the two dimensional side view shown in FIG. 14 also corresponds to the time at which the instrument is sounded, similar to the manner in which music is displayed in standard notation (to some degree). Thus, the visual representation of rhythm generated by the disclosed system and method can be easily converted to sheet music in standard notation by substituting the various bars (and spaces therebetween) into their corresponding representations in standard notation. For example, bar 1404 (representing the bass drum) will be converted to a note 1254 in the lowest space 1260a of staff 1252. Likewise, bar 1410 (representing the snare drum) will be converted to a note 1256 in the second highest space 1260c of staff 1252.

The 3-D visualization of this Rhythmical Component as shown, for example, in FIG. 12, results in imagery that appears much like a ‘wormhole’ or tube. For each composition of music, a finite length tube is created by the system which represents all of the rhythmic structures and relationships within the composition. This finite tube may be displayed to the user in its entirety, much like traditional sheet music. For longer compositions, the tube may be presented to the user in sections to accommodate different size video display screens. To enhance the user's understanding of the particular piece of music, the 3-D ‘wormhole’ image may incorporate real time animation, creating the visual effect of the user traveling through the tube. In one embodiment, the rhythmic structures appear at the point “nearest” to the user as they occur in real time, and travel towards the “farthest” end of the tube, giving the effect of the user traveling backwards through the tube.

The two-dimensional view of FIG. 13 can also be modified to incorporate a perspective of the user looking straight “into” the three-dimensional tube or tunnel, with the graphical objects made to appear “right in front of” the user and then move away and into the tube, eventually shrinking into a distant center perspective point. It shall be understood that animation settings for any of the views in FIGS. 12-14 can be modified by the user in various embodiments, such as reversing the animation direction or the duration of decay for objects which appear and the fade into the background. This method of rhythm visualization may also incorporate the use of color to distinguish the different rhythmic structures within a composition of music, much like the MASTER KEY™ diagrams use color to distinguish between tonal intervals. For example, each instance of the bass drum being sounded can be represented by a sphere of a given color to help the user visually distinguish it when displayed among shapes representing other instruments.

In other embodiments, each spheroid (whether it appears as such or as a circle or line) and each toroid (whether it appears as such or as a ring, line or bar) representing a beat when displayed on the graphical user interface will have an associated small “flag” or access control button. By mouse-clicking on one of these access controls, or by click-dragging a group of controls, a user will be able to highlight and access a chosen beat or series of beats. With a similar attachment to the Master Key™ music visualization software (available from Musical DNA LLC, Indianapolis, Ind.), it will become very easy for a user to link chosen notes and musical chords with certain beats and create entire musical compositions without the need to write music using standard notation. This will allow access to advanced forms of musical composition and musical interaction for musical amateurs around the world.

The present disclosure utilizes the previously described visualization methods as a basis for a system for providing medical treatment. The easily visualized tonal and rhythmic shapes provide a much more intuitive graphical format for purposes of interpreting the characteristics of signals used to detect and locate entities in the human body, such as kidney stones or tumors.

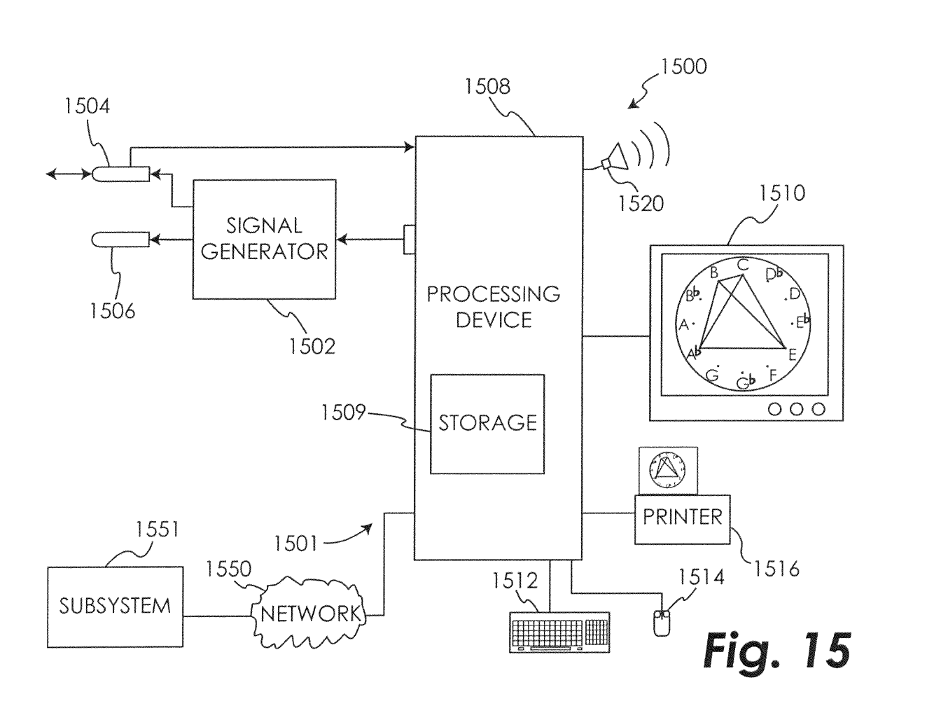

FIG. 15 shows, in schematic form, one embodiment of a medical treatment system 1500 according to the present disclosure. It is understood that one or more of the functions described herein may be implemented as either hardware or software, and the manner in which any feature or function is described does not limit such implementation only to the manner or particular embodiment described. The system 1500 may include an signal generator 1502, a diagnostic probe 1504, a treatment probe 1506, a processing device 1508, a data storage device 1509, a display 1510, user input devices such as keyboard 1512 and mouse 1514, a printer 1516, and one or more speakers 1520. These devices are operatively coupled to allow the transmission of signals between the devices so that the reflected diagnostic signals can be processed by the processing device 1502 and evaluated and/or manipulated by the user.

Signal generator 1502 is capable of creating various tones and rhythms at frequencies that span the audio and ultrasonic spectrums, such as pure or sine waves, square wave tones, multiple harmonic tones, and percussive sounds, as several non-limiting examples. Diagnostic probe 1504 comprises both emission and reception capabilities, with treatment probe 1506 being operable to produce signals capable of shattering kidney stones or other problematic entities.

The processing device 1508 may be implemented on a personal computer, a workstation computer, a laptop computer, a palmtop computer, a wireless terminal having computing capabilities (such as a cell phone having a Windows CE or Palm operating system), an embedded processor system, or the like. It will be apparent to those of ordinary skill in the art that other computer system architectures may also be employed.

In general, such a processing device 1508, when implemented using a computer, comprises a bus for communicating information, a processor coupled with the bus for processing information, a main memory coupled to the bus for storing information and instructions for the processor, a read-only memory coupled to the bus for storing static information and instructions for the processor. The display 1510 is coupled to the bus for displaying information for a computer user and the user input devices 1512 and 1514 are coupled to the bus for communicating information and command selections to the processor. A mass storage interface for communicating with data storage device 1509 containing digital information may also be included in processing device 1508 as well as a network interface for communicating with a network.

The processor may be any of a wide variety of general purpose processors or microprocessors such as the PENTIUM microprocessor manufactured by Intel Corporation, a POWER PC manufactured by IBM Corporation, a SPARC processor manufactured by Sun Corporation, or the like. It will be apparent to those of ordinary skill in the art, however, that other varieties of processors may also be used in a particular computer system. Display 1510 may be a liquid crystal device (LCD), a light emitting diode device (LED), a cathode ray tube (CRT), a plasma monitor, a holographic display, or other suitable display device. The mass storage interface may allow the processor access to the digital information in the data storage devices via the bus. The mass storage interface may be a universal serial bus (USB) interface, an integrated drive electronics (IDE) interface, a serial advanced technology attachment (SATA) interface or the like, coupled to the bus for transferring information and instructions. The data storage device 1509 may be a conventional hard disk drive, a floppy disk drive, a flash device (such as a jump drive or SD card), an optical drive such as a compact disc (CD) drive, digital versatile disc (DVD) drive, HD DVD drive, BLUE-RAY DVD drive, or another magnetic, solid state, or optical data storage device, along with the associated medium (a floppy disk, a CD-ROM, a DVD, etc.)

In general, the processor retrieves processing instructions and data from the data storage device 1509 using the mass storage interface and downloads this information into random access memory for execution. The processor then executes an instruction stream from random access memory or read-only memory. Command selections and information that is input at user input devices 1512 and 1514 are used to direct the flow of instructions executed by the processor. User input devices 1512 and 1514 may comprise a data entry keyboard, a mouse or equivalent trackball device, or electro-mechanical knobs and switches. The results of this processing execution are then displayed on display device 1510.

The processing device 1508 is configured to generate an output for viewing on the display 1510 and/or for driving the printer 1516 to print a hardcopy. Preferably, the video output to display 1510 is also a graphical user interface, allowing the user to interact with the displayed information.

The system 1500 may optionally include one or more remote subsystems 1551 for communicating with processing device 1508 via a network 1550, such as a LAN, WAN or the internet. Remote subsystem 1550 may be configured to act as a web server, a client or both and will preferably be browser enabled. Thus with system 1500, a user can provide medical diagnosis or treatment using system 1500 remotely.

In operation, signal generator 1502 provides a diagnostic signal to the diagnostic probe 1504. Diagnostic probe 1504 emits the signal which enters the patient's body in the vicinity of where a kidney stone (or other entity desired to be located) is believed to be. A portion of the signal is reflected back and is captured by the diagnostic probe 1504. Diagnostic probe 1504 then outputs the returned or reflected signal to processing device 1508. Processing device 1508 creates tonal and visual components from the received signals and outputs the result on display 1510. The tonal visualization components illustratively comprise the visual representation of the composition of the received signals, including, but not limited to, pitch, amplitude, and phase. A kidney stone, having a density and structure significantly different than the surrounding tissue, will absorb and reflect different portions of the energy or frequency spectrum of the signals output by the diagnostic probe 1504. Based on the appearance of the visualization components as diagnostic probe 1504 is moved slowly about the suspected kidney stone location, the user or automated processing software can determine the exact location of the stone. For example, the amplitude or other properties of the reflected signal will vary at different “note” positions on the associated tonal circle as the probe is moved in relation to the stone.

Once the kidney stone or other entity has been identified, treatment probe 1506 is moved to a position adjacent to the stone's location via registration technology that is known in the art. Signal generator 1502 then produces a percussive signal, which is output by treatment probe 1506, at the appropriate frequency and energy level, as is known in the lithotripsy art, to shatter or break up the kidney stone.

In order to visualize the individual frequencies of input sounds, the system 1500 can implement software operating as an audio signal or note extractor. The audio extractor examines the signals received by the processing device 1508 and determines which primary frequencies are present. The frequency content is then mapped to certain colors and positions within a tonal circle or helix and displayed to the user. Various methods are known in the art for determining the frequency of an input signal including, but not limited to, frequency counters and band pass filters. Certain audio frequency extraction methods are also described in U.S. Patent Application Serial No. 61/025,374 filed Feb. 1, 2008 entitled “Apparatus and Method for Visualization of Music Using Note Extraction” which is hereby incorporated by reference in its entirety.

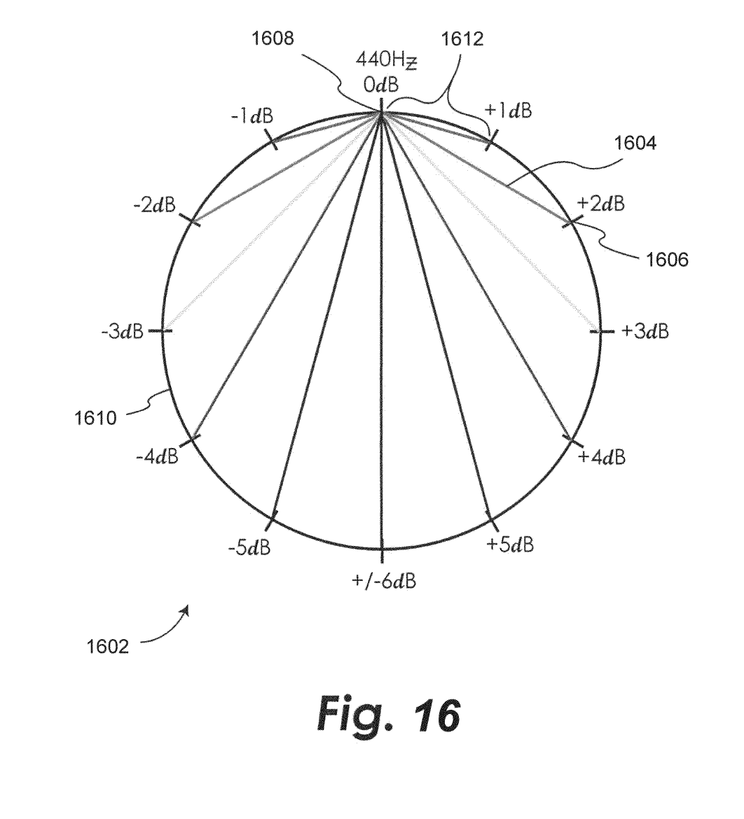

FIG. 16 shows a visualization scheme 1602 according to one embodiment where the color, representing amplitude for a given frequency, of each line 1604 is dependent on the deviation of the sensed amplitude 1606 from a reference or baseline amplitude 1608. FIG. 16 shows the various color gradations which correspond to different points or amplitudes along the circle 1610. As the sensed amplitude increases or decreases from the baseline amplitude 1608, the color of line 1604 will change according to the predefined scheme. As illustrated in FIG. 16, the color of lines 1604 changes from red to orange to yellow to green to blue to purple as the deviation increases. It shall be understood that any desired color scheme may be used.

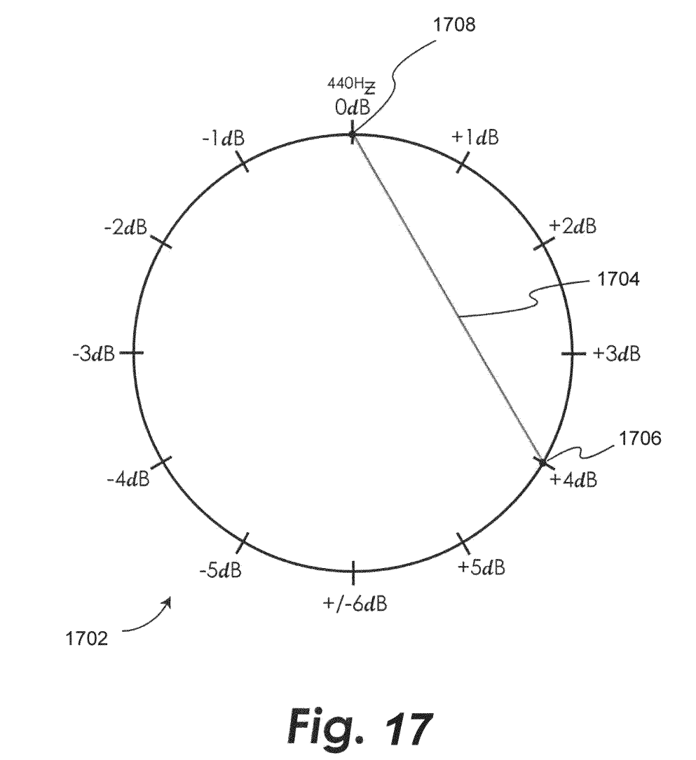

FIG. 17 shows one example where the baseline frequency is 440 Hz and the sensed amplitude at that frequency is approximately +4 decibels (dB) above the baseline amplitude 1708, resulting in a green line 1704 being displayed from indicator 1706 to the baseline amplitude 1708. For frequencies having amplitudes falling within the baseline amplitude 1708 and an immediately adjacent amplitude subdivision, an additional repeating rainbow can be displayed within the interval (indicated as 1612 on FIG. 16) to provide more guidance for the user.

The degree of accuracy in the visualization 1602 can be adjusted by the user. For example, if the sensed amplitude is within interval 1612, the user can select the visualization 1602 using the mouse 1514 or other input device, whereby the system 1500 will display a new visualization with smaller amplitude gradations. This technique is described further in U.S. Provisional Patent Application Serial No. 61/025/542 filed Feb. 1, 2008 entitled “Apparatus and Method of Displaying Infinitely Small Divisions of Measurement” which is herein incorporated by reference in its entirety.

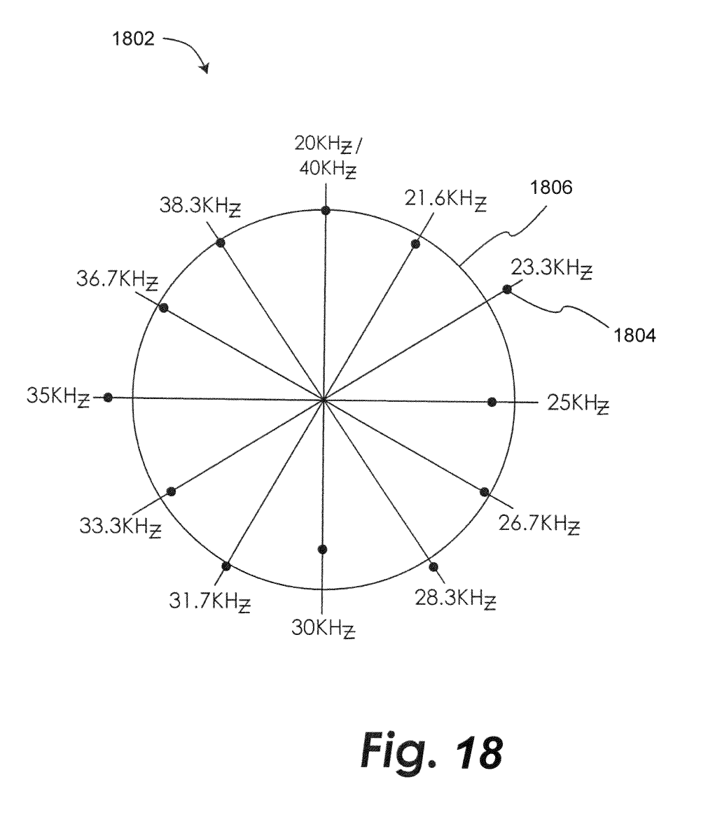

FIG. 18 illustrates a visualization created by processing device 1508 according to another embodiment. A tonal circle 1802 is subdivided into a number of frequency intervals determined by the desired accuracy. At each interval, an indicator 1804 is displayed which represents a given frequency. The amplitude of the signal at the given frequency corresponds to the radial distance of the indicator from a reference perimeter 1806. As the amplitude increases or decreases, the indicator will move radially outward or inward respectively. For example, as shown in FIG. 18, there is a higher amplitude at the 23.3 KHz frequency and a lower amplitude at the 25 KHz frequency. This visualization can be further extended by displaying the circle as a continuous helix upon which the various amplitude indicators are displayed.

In addition to amplitude, other signal characteristics can be displayed using the method of the present disclosure. For example, the signal phase in relation to an established time reference can be displayed using the circular representations discussed above. Information concerning the amount of compression or limiting can also be displayed, along with data representing thresholds, rates, attacks, and release.

Medical treatment system 1500 therefore provides a dual function of locating and treating a kidney stone without subjecting a patient to harmful X-radiation. While the disclosure has been illustrated and described in detail in the drawings and foregoing description, the same is to be considered as illustrative and not restrictive in character, it being understood that only the preferred embodiments have been shown and described and that all changes, modifications and equivalents that come within the spirit of the disclosure provided herein are desired to be protected. The articles “a,” “an,” “said,” and “the” are not limited to a singular element, and may include one or more such elements.

What is claimed:

1. A system for providing medical treatment of kidney stones, comprising:
an signal output source;
a signal input device;
a processing device; and
a display,
wherein:
said signal output source directs a first output signal in the vicinity of an entity in a human body desired to be treated;
wherein:
said processing device executes computer readable code to create a first visual representation of a first signal reflected by said entity and received by said signal input device for output on said display; and
wherein:
said first visual representation is generated according to a method comprising the steps of:
(a) labeling the perimeter of a circle with a plurality of labels corresponding to a plurality of frequency bands, such that moving radially inward or outward from any one of said labels represents a change in a signal amplitude at the frequency corresponding to said one of first labels;
(b) identifying a first occurrence of a first frequency having a first amplitude within said first signal; and
(c) graphically indicating a point along a radial axis corresponding to said first amplitude; said radial axis connecting the center of said circle and said first label.
